(19) United States
(12) Patent Application Publication
(10) Pub. No.: US 2025/0328591 A1
(43) Pub. Date: Oct. 23, 2025

(54) SYSTEMS AND METHODS FOR INTERACTING WITH KNOWLEDGE GRAPHS

(71) Applicant: The Florida State University Research Foundation, Inc., Tallahassee, FL (US)

(72) Inventor: Mikhail Gubanov, Tallahassee, FL (US)

(21) Appl. No.: 19/039,250

(22) Filed: Jan. 28, 2025

Related U.S. Application Data
(60) Provisional application No. 63/626,261, filed on Jan. 29, 2024.
(60) Provisional application No. 63/709,723, filed on Oct. 21, 2024.

(51) Int. Cl. G06F 16/9535; G06F 16/9538; G06N 5/022

(52) U.S. Cl. CPC G06F 16/9535; G06F 16/9538; G06N 5/022

(57) ABSTRACT

A method including: displaying, on a graphical user interface, a knowledge graph associated with a domain, wherein the knowledge graph includes a number of nodes and a number of edges representing relationships between the number of nodes, wherein the number of nodes include a number of leaf nodes, each of the number of leaf nodes being associated with respective metadata related to the domain; receiving, at the graphical user interface, one or more user inputs, wherein the one or more user inputs include a selection of a specific leaf node of the number of leaf nodes; displaying, on the graphical user interface, the respective metadata related to the domain that is associated with the specific leaf node; and providing, on the graphical user interface, a search window configured to receive a search query related to the domain.

### CROSS-REFERENCE TO RELATED APPLICATIONS

This application claims the benefit of and priority to U.S. Provisional Patent Application No. 63/626,261, filed on Jan. 29, 2024, and U.S. Provisional Patent Application No. 63/709,723, filed on Oct. 21, 2024, the entire contents of each of which are incorporated herein by reference.

### STATEMENT REGARDING FEDERALLY SPONSORED RESEARCH OR DEVELOPMENT

This invention was made with government support under award number 2345794 awarded by the National Science Foundation. The government has certain rights in the invention.

### BACKGROUND

Medical knowledge may increase rapidly. For example, published peer-reviewed medical knowledge may double every few months. This rapid increase may make it difficult to access new and/or existing medical knowledge (e.g., thereby hindering awareness of the latest best practices for patients, their families, and medical professionals). Moreover, conventional solutions for locating, collecting, and/or processing medical knowledge may be prohibitively slow (e.g., because of inconvenient user interfaces, a lack of useful filtering, etc.).

Thus, a solution for providing access to and interacting with the latest, trustworthy, medical findings is desirable. The present disclosure relates generally to the field of knowledge graph data structures, and more specifically to systems and methods of generating a hybrid knowledge graph-large language model data structure to surface and/or present information to a user.

### SUMMARY

One implementation of the present disclosure is a method including: displaying, on a graphical user interface, a knowledge graph associated with a domain, wherein the knowledge graph includes a number of nodes and a number of edges representing relationships between the number of nodes, wherein the number of nodes include a number of leaf nodes, each of the number of leaf nodes being associated with respective metadata related to the domain; receiving, at the graphical user interface, one or more user inputs, wherein the one or more user inputs include a selection of a specific leaf node of the number of leaf nodes; displaying, on the graphical user interface, the respective metadata related to the domain that is associated with the specific leaf node; and providing, on the graphical user interface, a search window configured to receive a search query related to the domain.

In some embodiments, the method further includes: receiving, at the search window, the search query; executing, using a search engine, the search query against the respective metadata related to the domain that is associated with the specific leaf node; and returning, in response to the search query, a subset of the respective metadata related to the domain that is associated with the specific leaf node. In some embodiments, the search engine is a keyword search engine. In some embodiments, the search engine is a structural search engine. In some embodiments, the search engine is a large language model (LLM) search engine. In some embodiments, the method further includes generating a three-dimensional (3D) meta-profile for the respective metadata related to the domain that is associated with the specific leaf node. In some embodiments, the method further includes displaying, on the graphical user interface, the 3D meta-profile. In some embodiments, the method further includes constructing the knowledge graph. In some embodiments, the steps of constructing the knowledge graph include: initializing a structural hierarchy of the knowledge graph based, at least in part, on a user specification; and automatically fusing the respective metadata related to the domain to each of the number of leaf nodes. In some embodiments, the domain is cancer.

Another implementation of the present disclosure is a system including: a computing cluster including a number of computing devices, each computing device including at least one processor and a memory operably coupled to the at least one processor; a database operably coupled to the computing cluster, wherein the computing cluster is configured to: display, on a graphical user interface, a knowledge graph associated with a domain, wherein the knowledge graph includes a number of nodes and a number of edges representing relationships between the number of nodes, wherein the number of nodes include a number of leaf nodes, each of the number of leaf nodes being associated with respective metadata related to the domain; receive, at the graphical user interface, one or more user inputs, wherein the one or more user inputs include a selection of a specific leaf node of the number of leaf nodes; display, on the graphical user interface, the respective metadata related to the domain that is associated with the specific leaf node; and provide, on the graphical user interface, a search window configured to receive a search query related to the domain

Another implementation of the present disclosure is a method for presenting information to a user. In some embodiments, the method includes initializing a graph data structure using a seed to generate an initialized graph data structure. In some embodiments, the method includes training a machine learning (ML) model using a corpus to generate a hierarchical data structure including a subtree extracted from the corpus. In some embodiments, the method includes updating the initialized graph data structure using the hierarchical data structure by adding at least one of (i) a node or (ii) an edge to the initialized graph data structure to generate an updated graph data structure, wherein the node or the edge is a representation of at least a portion of the subtree.

In some embodiments, generating the hierarchical data structure includes analyzing the corpus using a large language model (LLM) to produce a topical table cluster, wherein the subtree is associated with a cluster of the topical table cluster. In some embodiments, analyzing the corpus includes generating at least two embedding vectors based on the corpus, generating a centroid vector based on the initialized graph data structure, and comparing the at least two embedding vectors to the centroid vector to identify an embedding vector of the at least two embedding vectors that is within a threshold degree from the centroid vector. In some embodiments, the threshold degree is 18 degrees.

In some embodiments, the corpus is represented in a JavaScript Object Notation (JSON) format. In some embodiments, updating the initialized graph data structure using the hierarchical data structure includes identifying a node of the initialized graph data structure that corresponds to a node of the subtree. In some embodiments, the method further includes receiving a query for information, traversing the updated graph data structure to identify a node associated with the query, and transmitting information associated with the identified node. In some embodiments, transmitting the information associated with the identified node includes displaying a graphical user interface (GUI) that represents the identified node. In some embodiments, a user can traverse the updated graph data structure using the GUI. In some embodiments, receiving the query includes performing natural language processing (NLP) on a string. In some embodiments, transmitting the information associated with the identified node includes displaying metadata associated with a table associated with the identified node. In some embodiments, transmitting the information associated with the identified node includes displaying a table to a user.

In some embodiments, the corpus includes a peer-reviewed publication. In some embodiments, the method further includes generating a confidence score associated with adding the node or the edge to the initialized graph data structure by comparing a degree of separation between (i) the node or the edge and (ii) a node of the initialized graph data structure, comparing the confidence score to a threshold, and in response to the comparison, surfacing the node or the edge for review.

Another implementation of the present disclosure includes a graphical user interface (GUI) for retrieving medical information. The GUI may be configured to receive a natural language text input from a user, transmit, to a computing system, the natural language text input for processing, wherein the computing system processes the natural language text input by (i) tokenizing the natural language text input to generate a query and (ii) searching a corpus using the query to identify a table, receive, from the computing system, the table, and display the table to the user.

In some embodiments, searching the corpus includes searching a first document and a second document and ranking the first document and the second document based on a term frequency-inverse document frequency (TF-IDF). In some embodiments, searching the first document includes comparing the query to a field of the document, wherein the field includes at least one of a title, an abstract, body text, table captions, table data, metadata, figure captions, or figure content. In some embodiments, searching the first document includes comparing the query to the title, the abstract, the body text, the table captions, the table data, the metadata, the figure captions, and the figure content. In some embodiments, searching the corpus includes at least one of (i) hierarchical vertical and horizontal schema matching, (ii) data transformation and unification, (iii) processing nested tables inside cells, or (iv) ranking search results by relevance.

### BRIEF DESCRIPTION OF THE DRAWINGS

The above and other aspects and features of the present disclosure will become more apparent to those skilled in the art from the following detailed description of the example embodiments with reference to the accompanying drawings.

FIG. 1 is a block diagram of an environment for interacting with knowledge graphs, according to an exemplary embodiment.

FIG. 2 is a flow diagram illustrating example operations for interacting with knowledge graphs, according to an exemplary embodiment.

FIG. 3 is a fragment of an example knowledge graph, according to an exemplary embodiment.

FIG. 4 is a graphical user interface including search and results windows, according to an exemplary embodiment.

FIG. 5 is a 3D meta-profile, according to an exemplary embodiment.

FIG. 6 is a flow diagram illustrating a method of presenting information to a user, according to an exemplary embodiment.

FIG. 7 is a neural network, according to an exemplary embodiment.

FIG. 8 is a computing device, according to an exemplary embodiment.

### DETAILED DESCRIPTION

Domain-specific information may expand rapidly. For example, published peer-reviewed medical knowledge and practices may double every few months. This may complicate access to information, thereby making it difficult for parties to stay up to date on the latest best practices. Moreover, this may result parties resorting to inefficient searching/reading/filtering to obtain relevant information. The present disclosure relates to a hybrid knowledge graph (KG)-large language model (LLM) that may provide improved access to information. At various points this disclosure may use the term “graph data structure” to refer to a knowledge graph. In various embodiments, the system is a retrieval augmented generation (RAG)-based system including a LLM moderated by a KG. This hybrid may result in improved verifiability and compatibility with multi-modal content. Conventional KGs, deep-learning models, and/or LLMs may have difficulty reliably retrieving and organizing complex knowledge from thousands of publications, without significant human supervision to ensure correctness. Moreover, conventional LLMs may suffer from limitations, such as “hallucinations” and “catastrophic forgetting” which may result in “forgetting” important information or inventing fake facts. Furthermore, conventional LLMs may be trained on outdated data (e.g. training data having a cut-off date of September 2021, etc.) and/or may be prohibitively expensive to retrain. Additionally, manually maintained KGs may become stale or may have limited coverage.

Systems and method of the present disclosure may offer many benefits. For example, a RAG-based hybrid may scale to thousands of data sources, “understand” multi-modal knowledge, be robust against hallucination, and may not require significant supervision. Moreover, the RAG-based hybrid may learn from publications (e.g., publications from PubMed.com, etc.). In various embodiments, the RAG-based hybrid may include several interactive interfaces. For example, the RAG-based hybrid may include a browsing interface, a search interface, and a natural language interface. The present disclosure describes the RAG-based hybrid in the context of medical information; however, it should be understood that the RAG-based hybrid can be applied to other domains and the present disclosure is not meant to be limiting.

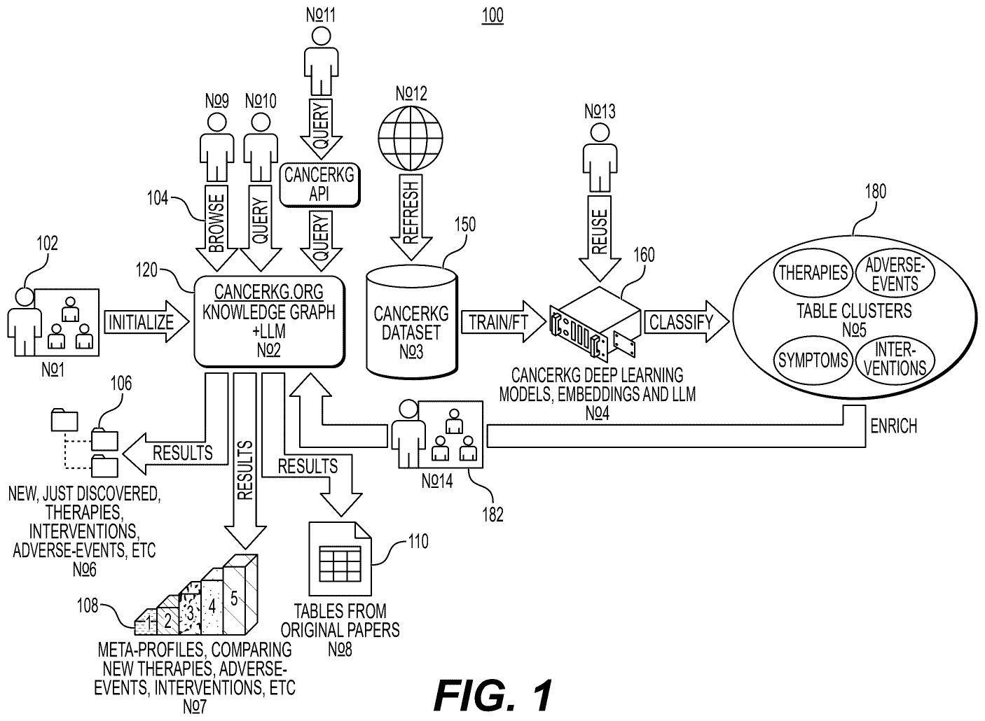

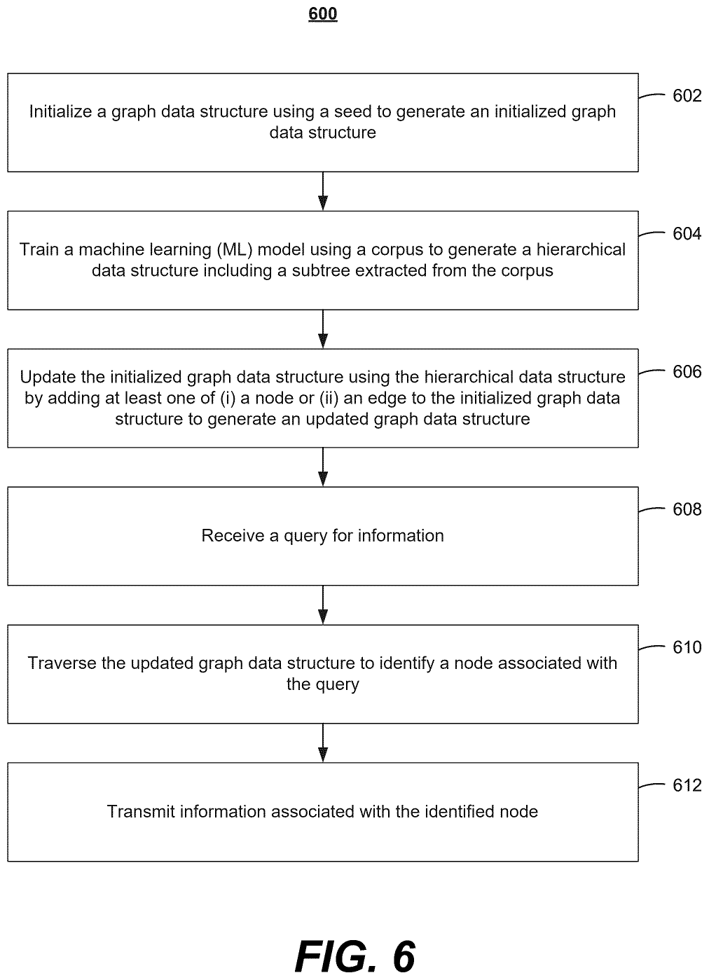

Referring now to FIG. 1, environment 100 for interacting with knowledge graphs is shown. An example system can include processing system 160. Processing system 160 may be and/or include a computing cluster (e.g., implemented as a server, etc.) including a number of computing devices. Each of the computing device includes at least one processor and a memory operably coupled to the at least one processor (e.g., the basic configuration of line 802 in FIG. 6). Additionally, the system can include a database (e.g. a sharded MongoDB) operably coupled to processing system 160. The database stores dataset 150 related to a specific domain. An example domain is cancer such as colorectal cancer. It should be understood that cancer is provided only as an example domain. This disclosure contemplates interacting with knowledge graphs related to domains other than cancer. In some embodiments, the system is a RAG-based system.

In various embodiments, a user may initialize 102 knowledge graph 120. For example, the user may initialize a knowledge graph with 10-20 nodes to serve as a seed knowledge graph. In various embodiments, knowledge graph 120 is interactive and may be browsed and/or queried. For example, knowledge graph 120 may be queried using a publication, a table structural search-engine, and/or an API. In various embodiments, dataset 150 may be parsed, post-processed, and/or restructured before storage in a semi-structured format. For example, dataset 150 may be stored in a JavaScript object notation (JSON) format. Processing system 160 may perform training, classifying, clustering, and/or fine-tuning associated with the LLM. Additionally or alternatively, processing system 160 may receive and respond to queries for information. In various embodiments, processing system 160 generates one or more table clusters 180 based on dataset 150. In various embodiments, processing system 160 generates one or more hierarchical knowledge graph fragments 106 based on table clusters 180.

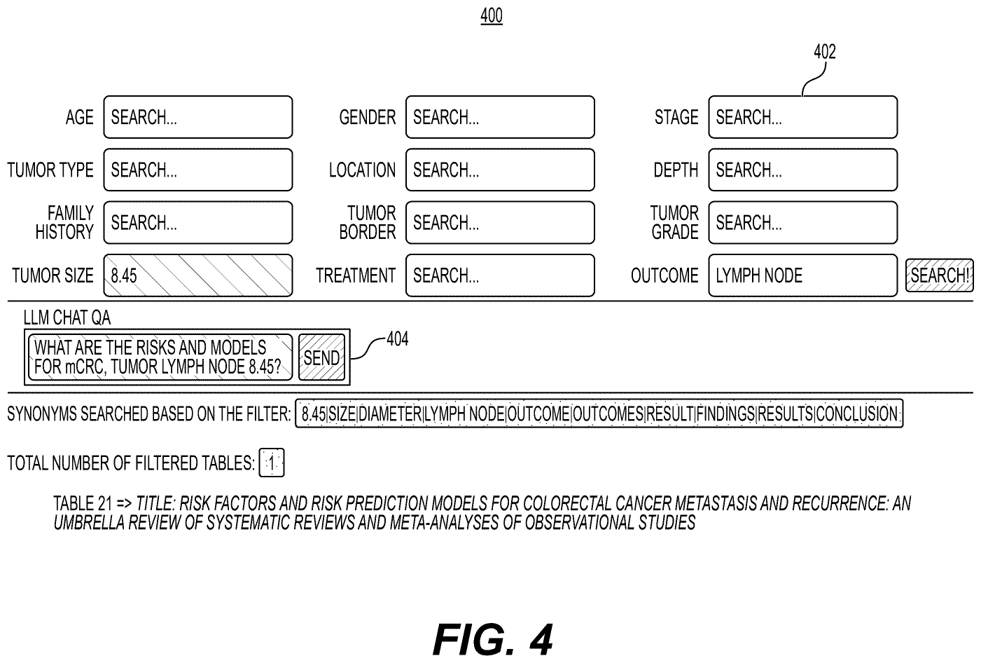

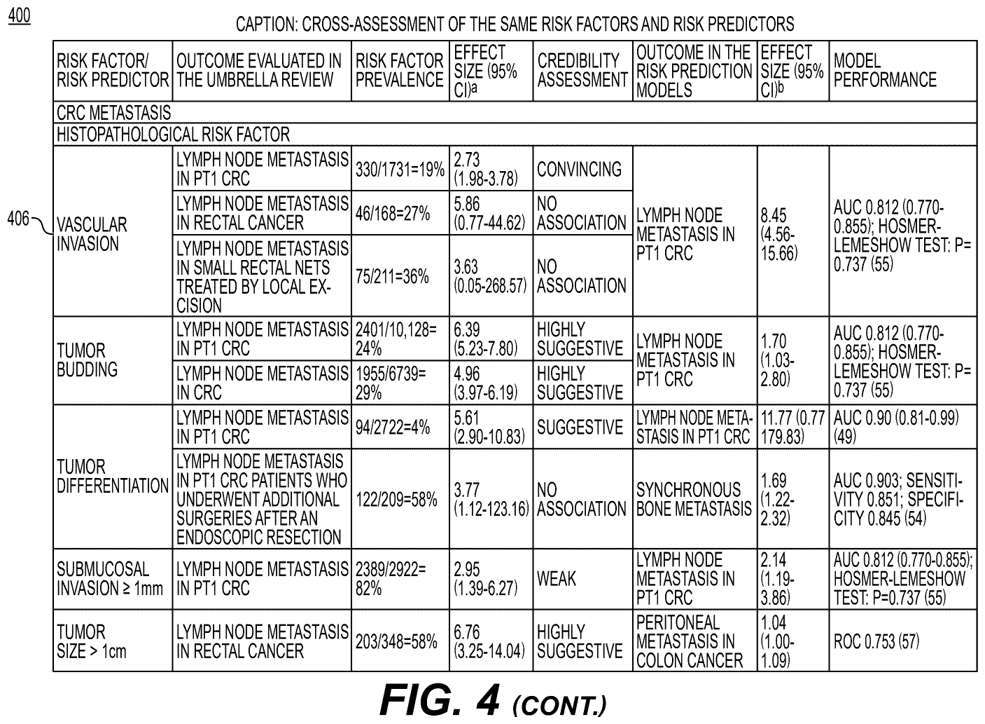

A user may interact 104 with knowledge graph 120. For example, a user may query knowledge graph 120 to retrieve data demonstrating the efficacy of different cancer therapies. In some embodiments, users query knowledge graph 120 using an application programming interface (API) (shown as No. 11). For example, user may query knowledge graph 120 using RPC and/or REST calls. Information from knowledge graph 120 may be presented to users in various formats. For example, information may be presented via multi-layered 3D metaprofiles 108, tables 110, and/or via a conversational interface (shown as No. 10). Multi-layered 3D metaprofiles 108 may be or include a visualization/browsing interface for viewing large topical table clusters (e.g., as shown in FIG. 4 below, etc.). A topical table cluster may be or include a set of tables associated with a topic. For example, a number of tables including songs. In various embodiments, knowledge graph 120 stores tables extracted from the training corpus. Tables 110 stored in knowledge graph 120 may be presented to users. In various embodiments, dataset 150 is updated with new information (shown as No. 12). For example, dataset 150 may be updated with information from PubMed.com. In various embodiments, knowledge graph 120 is enriched based on the updated information (shown as update 182). For example, knowledge graph 120 may be enriched through fusion of new knowledge graph sub-trees and/or insertion of new nodes/edges.

In various embodiments, information used to update dataset 150 is processed before adding it to dataset 150. For example, the information may be processed by encoding numerical data using regular expressions. In various embodiments, generating table clusters includes identifying a representative (e.g., centroid) table associated with a topic. The representative table may be used as a seed to train a gated recurrent unit (GRU) binary classification model and/or create a cluster for the topic. In various embodiments, generating table clusters includes generating an embedding vector corresponding to each centroid table. Each table vector may include VHMD for horizontal metadata (HMD), VVMD for vertical metadata (VMD), VD for data (D). Each of them may be calculated as a summation of the embedding vectors associated with each term located in the tables.

A final embedding vector for a table may be V_{T}=V_{HMD}+V_{VMD}+V_{D}. VMD may refer to vertical metadata which may be or include portions of a table having attributes but no data. In various embodiments, generating table clusters includes generating a centroid vector and selecting one or more tables from the dataset within a degree of the centroid vector. For example, tables within 18 degrees of the centroid vector may be selected. In various embodiments, generating table clusters includes training the GRU model as a binary topic classifier using the selected on the or more tables. A centroid vector may be or include a vector representing a semantic center of a set of vectors pointing in approximately the same direction in the vector space. In some embodiments, a centroid vector is calculated as a mean/average of these vectors. However, it should be understood that a centroid vector may be calculated in other manners as well. In some embodiments, the one or more tables are appended with random tables from the dataset. Table clusters may be formed by running all such topical binary classifiers through the dataset.

In various embodiments, once the knowledge graph 120 is initialized, extracted information from dataset 150 is added/fused into knowledge graph 120 (e.g., the enrichment process). Clusters may be classified and extracted. A user may search knowledge graph 120 via an interface (e.g., as shown in FIG. 4 below). In various embodiments, matching nodes and/or paths leading to matching nodes are highlighted. A user may browse knowledge graph 120 to explore table clusters attached to the nodes. Additionally or alternatively, a user may select a cluster to query tables 110 in the cluster and/or generate meta-profiles 108 corresponding to the cluster.

In various embodiments, fusing extracted hierarchical knowledge into knowledge graph 120 includes matching a root node of an extracted subtree to one or more nodes in knowledge graph 120. The matching process may be based on normalized NLP term matching, amended by the embedding-driven matching. Embedded-driven matching may facilitate matching of new terms. For example, an extracted subtree may be: “2^{nd} line treatments→Regorafenib” (e.g., as extracted from table metadata). The root node “therapy” may match to a KG node “therapy(ies)” by normalized NLP term matching and the leaves (“Regorafenib”) may be merged with the leaves of the matched node in the KG. However, if there is no corresponding KG node “therapy(ies)” and there is no match to the KG leaves with existing therapies, the embedding vector corresponding to the new therapy (“Regorafenib”) extracted from metadata may be used to match it to the embedding vectors of the existing therapies in knowledge graph 120 (e.g., based on their proximity to one another). The node “therapy” may then be added to knowledge graph 120 on the top of the “Regorafenib” node. As a further example, if the extracted subtree has several layers of hierarchy (e.g., “side-effects→pediatric side-effects→severe pain”), it may be left separate from the existing side-effects in knowledge graph 120 (even if matched to them by having close embedding vectors because it is categorized as “pediatric side effects,” which is a separate category from “regular side-effects,” so both the new node Pediatric side-effects and its leaves have to be added to knowledge graph 120, even if some of the side-effects overlap with the general side-effects already present in knowledge graph 120). In some embodiments, fusion of sub-trees having several layers and/or insertion of new nodes matching with a low confidence score are identified for validation (e.g., by a user, etc.). In some embodiments, fusion of leaves with nodes matched with high confidence score are unsupervised.

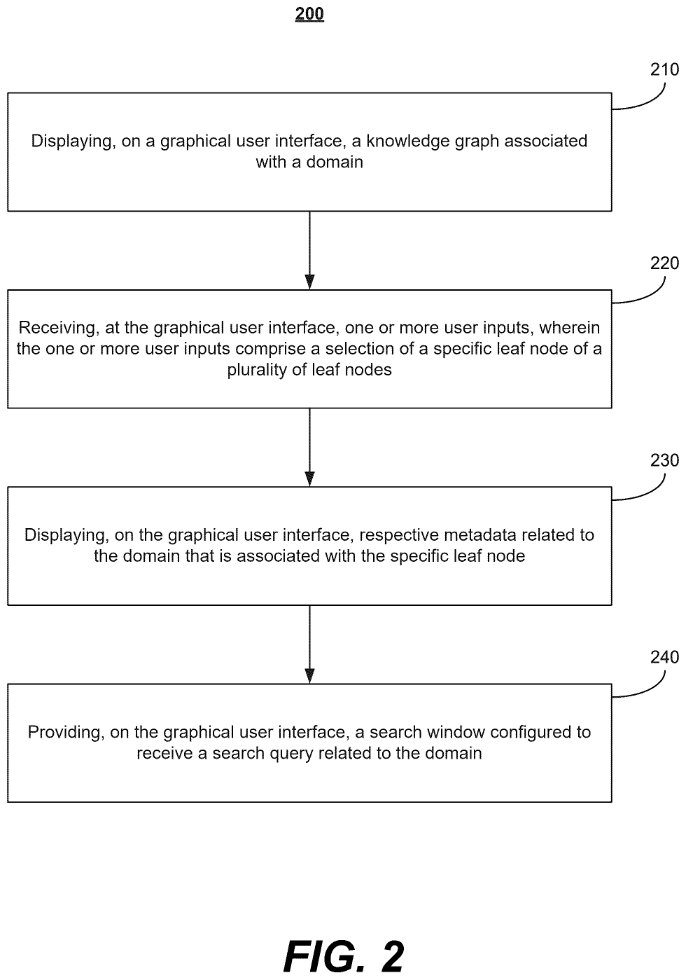

Referring now to FIG. 2, a flowchart of an example method 200 for interacting with knowledge graphs is shown. In various embodiments, the system of FIG. 1 performs method 200. For example, processing system 160 may perform method 200.

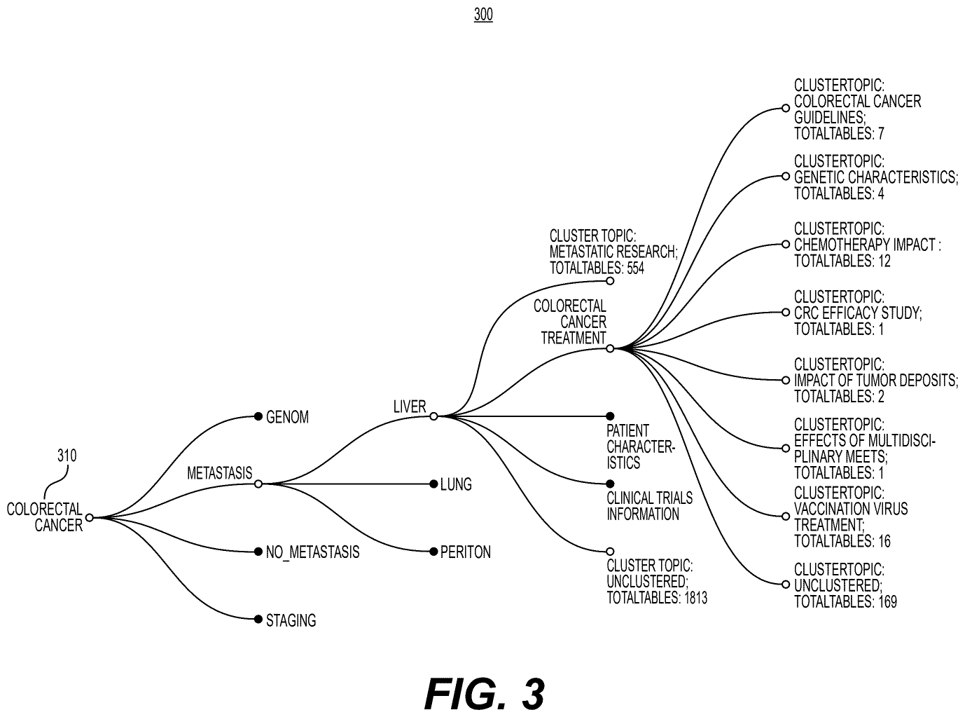

At step 210, method 200 includes displaying, on a graphical user interface, a knowledge graph associated with a domain. For example, the domain may be colorectal cancer or any other domain. A knowledge graph is structured to model complex relationships and information in a way that is easily traversable and understandable. For example, a knowledge graph includes a number of nodes and a number of edges. The nodes represent concepts in the knowledge graph. The edges connect nodes and represent relationships between them. These relationships define how different nodes are connected or related to each other. Additionally, the number of nodes include a number of leaf nodes, which refer to nodes that do not have any outgoing edges, meaning leaf nodes are not connected to any other nodes in the graph. Leaf nodes represent the most specific or granular concepts in the knowledge graph. The leaf nodes are associated with specific information or attributes about a particular concept. For example, each of the number of leaf nodes is associated with respective metadata related to the domain of colorectal cancer. To continue the example, the metadata may be extracted structured data (e.g. tables) from articles related to the domain. Alternatively or additionally, the metadata may be extracted text associated with figures from articles related to the domain. Optionally, the articles are peer-reviewed articles. It should be understood that tables and figure text are provided only as examples of metadata. Alternatively or additionally, this disclosure contemplates using machine learning models such as a large language model (LLM) to convert unstructured text from articles into structured text, which can be extracted as a form of metadata. A fragment of an example knowledge graph is shown in FIG. 3. The knowledge graph of FIG. 3 is related to colorectal cancer, which is the domain.

In some implementations, method 200 optionally includes constructing the knowledge graph. For example, the steps of constructing the knowledge graph include: initializing a structural hierarchy of the knowledge graph based, at least in part, on a user specification; and automatically fusing the respective metadata related to the domain to each of the plurality of leaf nodes.

At step 220, method 200 includes receiving, at the graphical user interface, one or more user inputs. For example, referring now to FIG. 3, a user may first select (e.g. a user input) node 310 labeled “colorectal cancer” to expose a layer of nodes including nodes labeled “genom,” “metastasis,” “no_metastasis,” and “staging,” all of which were previously hidden. Thereafter, the user may select (e.g. a user input) the node labeled “metastasis” to expose a layer of nodes including nodes labeled “liver,” “lung,” and “periton,” all of which were previously hidden. As shown in FIG. 3, the user may continue to select (e.g. a user input) nodes of interest until a number of leaf nodes related to “colorectal cancer treatment” are exposed. As discussed above, each of the number of leaf nodes is associated with respective metadata related to “colorectal cancer treatment.” Finally, the user may select (e.g. a user input) a specific leaf node of the number of leaf nodes.

At step 230, method 200 includes displaying, on the graphical user interface, the respective metadata related to the domain that is associated with the specific leaf node. For example, the metadata related to “colorectal cancer treatment” can be displayed on the graphical user interface. Results window 406 for displaying selected metadata of interest to the user is shown in FIG. 4, which illustrates an example implementation of a graphical user interface.

At step 240, method 200 includes providing, on the graphical user interface, a search window configured to receive a search query related to the domain. FIG. 4 illustrates an example implementation including structured search window 402 and LLM search window 404.

In some implementations, method 200 further includes: receiving, at the search window, the search query; executing, using a search engine, the search query against the respective metadata related to the domain that is associated with the specific leaf node; and returning, in response to the search query, a subset of the respective metadata related to the domain that is associated with the specific leaf node. In some implementations, the search engine is a keyword search engine. In some implementations, the search engine is a structural search engine. In some implementations, the search engine is a large language model (LLM) search engine. Optionally, a number of search windows can be provided, each being associated with a different search engine (e.g., structured search window 402 and LLM search window 404, etc.).

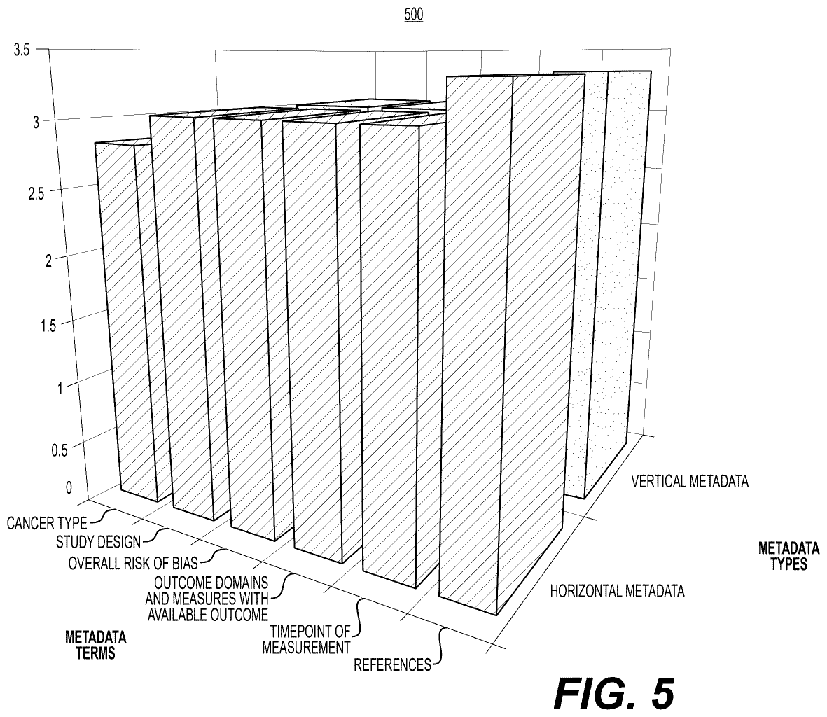

In some implementations, method 200 further includes generating a three-dimensional (3D) meta-profile for the respective metadata related to the domain that is associated with the specific leaf node. Optionally, method 200 further includes displaying, on the graphical user interface, the 3D meta-profile. An example 3D meta-profile is shown in FIG. 5.

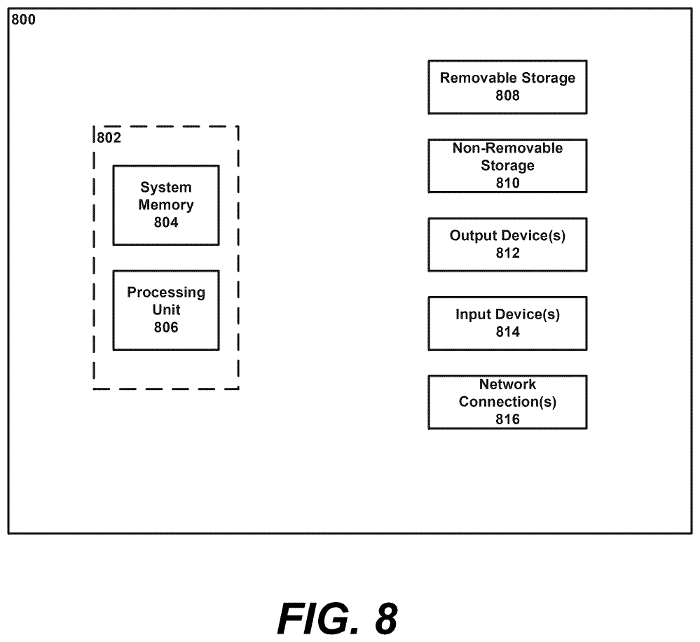

It should be appreciated that the logical operations described herein with respect to the various figures may be implemented (1) as a sequence of computer implemented acts or program modules (i.e., software) running on a computing device (e.g., the computing device described in FIG. 8), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring now to FIG. 3, knowledge graph 300 is shown, according to an exemplary embodiment. In various embodiments, the system of FIG. 1 generates knowledge graph 300. Knowledge graph 300 may include one or more node(s) 310. Knowledge graph 300 may represent hierarchical information for efficient search and retrieval by a user. In some embodiments, a user can interact with knowledge graph 300. For example, a user may click on a first node 310 and follow the unfolding path through various other nodes (e.g., colorectal cancer, metastasis, liver, colorectal cancer treatment, etc.).

Referring now to FIG. 4, interface 400 is shown, according to an exemplary embodiment. In various embodiments, interface 400 may be displayed to a user to facilitate querying the knowledge graph. In some embodiments, the system of FIG. 1 generates interface 400. Interface 400 may include structured search window 402, LLM search window 404, and/or results window 406. As shown, interface 400 displays search results for tables evaluating clinical outcomes with risk factors for colorectal cancer.

In various embodiments, queries received at LLM search window 404 may be tokenized. For example, the system may tokenize a search string and perform stemming on the tokenized string. Additionally or alternatively, queries are parsed (e.g., by a conversational query parser) into a number of queries. For example, the query shown in interface 400 may be split into a first query (e.g., a structural query that includes extracted attributes “lymph node” and “tumor size 8.45”) and a second query (e.g., a textual query that includes the input and synonyms for identified table fields in the input).

In various embodiments, LLM search window 404 presents a conversational interface for a user. For example, a user may input a question into LLM search window 404 and the system may parse the question (e.g., using the conversational query parser), identify any table attributes (and their values), automatically fill out the fields in structured search window 402, amend the tables (if any) (e.g., using a LLM that generates natural language amendments to the query), and perform the search.

Referring now to FIG. 5, 3D meta-profile 500 is shown, according to an exemplary embodiment. In various embodiments, 3D meta-profile 500 is displayed to a user in response to a user input (e.g., a query, etc.). For example, 3D meta-profile 500 may be displayed to a user in response to a user browsing a knowledge graph, drilling down to a “summaries and case studies” leaf node, and selecting an option “create 3D meta-profile” from a menu. As used herein a “meta-profile” is a summary of metadata of a table cluster. It should be understood that meta-profiles may take various forms and 3D meta-profile 500 is meant as a non-limiting example. 3D meta-profile 500 may include two or more axes. For example, 3D meta-profile 500 may include an x-axis (e.g., attribute labels of HMD of tables from the cluster, etc.), a y-axis (e.g., attribute labels of VMD of tables from the cluster, etc.), and a z-axis (e.g., a TF/IDF, score corresponding to each HMD or VMD attribute, etc.). In various embodiments, a user can interact with 3D meta-profile 500. For example, a user may select a bar to drill down to a subset of tables from the cluster specifically having only the selected attributes. In various embodiments, 3D meta-profile 500 functions as a dynamic filter (e.g., to facilitate surfacing relevant information by creating new table sub-clusters based on HMD and VMD choices). For example, if a user selects the “study design” bar, the system may generate a separate table sub-cluster having only the tables from the original cluster having “study design” in their HMD.

Referring now to FIG. 6, method 600 for presenting information to a user is shown, according to an exemplary embodiment. In various embodiments, the system of FIG. 1 performs method 600. For example, a processing circuit including a processor and memory may perform method 600.

At step 602, the system may initialize a graph data structure using a seed to generate an initialized graph data structure. In various embodiments, the seed may be and/or include a representative table (e.g., a centroid) for each topic. In some embodiments, the representative table is received/identified from/by a user.

At step 604, the system may train a machine learning (ML) model using a corpus to generate a hierarchical data structure including a subtree extracted from the corpus. In various embodiments, the corpus includes the representative tables from step 602. In various embodiments, step 604 includes training a GRU binary classifier using the representative table(s) and creating a cluster for each topic. In various embodiments, step 604 includes creating a composite embedding vector corresponding to each topical centroid table. Each table vector may include a number of components. For example, a first table vector may include a first term (e.g., V_{HMD} for HMD, etc.) a second term (e.g., V_{VMD} for VMD, etc.) and a third term (e.g., V_{D} for D, etc.). Each component may be calculated as a sum of embedding vectors. For example, the final embedding vector (V_{T}) for each table may be V_{T}=V_{HMD}+V_{VMD}+V_{D}. In various embodiments, step 604 includes selecting tables in the dataset within a threshold distance (e.g., 18 degrees, etc.) of a centroid vector. Step 604 may include training a GRU model as a binary topic classifier on the selected tables. Additionally or alternatively, step 604 may include training the GRU model on a number of random tables (e.g., from the dataset, etc.).

At step 606, the system may update the initialized graph data structure using the hierarchical data structure by adding at least one of (i) a node or (ii) an edge to the initialized graph data structure to generate an updated graph data structure. In various embodiments, step 606 includes executing the trained machine learning model (e.g., in inference mode, etc.) by using the topical binary classifiers to analyze the dataset and form table clusters. In various embodiments, step 606 includes fusing extracted information into the graph data structure. For example, the system may add nodes and edges corresponding to topics in a domain (e.g., topics in colorectal cancer, etc.). Fusing extracted information may include identifying a number of layers of abstraction. For example, in a medical context, “symptoms” may be a node in a subtree “clinical presentation” that may be linked to a “colorectal cancer” graph data structure root node. Fusion may include matching a root node (e.g., via normalized NLP term matching amended by embedding-driven matching, etc.) of an extracted subtree to a corresponding node in the graph data structure. In various embodiments, each fusion action is associated with a confidence score. In some embodiments, fusion actions having low confidence scores are surfaced for review (e.g., by a user, etc.). In various embodiments, the system learns from fusion mistakes over time and automatically corrects such mistakes. In some embodiments, the graph data structure is stored in JSON format, however other formats are possible.

At step 608, the system may receive a query for information. For example, the system may receive a natural language query such as “What are the risks and models for mCRC, tumor lymph node 8.45?” The query may be a structured query, a semi-structured query, and/or an unstructured query.

At step 610, the system may traverse the updated graph data structure to identify a node associated with the query. For example, the system may traverse the updated graph using a depth-first search, a breadth-first search, and/or any other algorithm for traversing a graph data structure. In various embodiments, step 610 includes highlighting a path to the node. In various embodiments, step 601 includes identifying a number of nodes. In various embodiments, the system may rank the results (e.g., the identified nodes, etc.). Ranking the results may include ranking based on (i) the number of matches, (ii) the proximity between matched terms, and/or (iii) the relative importance of the matched field/term and/or the like. The relative importance of the matched field/term may be based on a term frequency inverse document frequency (TF-IDF) weight associated with each term in the corpus. For example, each root form of a word in a corpus may be assigned a TF-IDF weight that indicates the importance of terms having that root form. To continue the example, documents may be ranked based on the TF-IDF weights in each document. In some embodiments, step 610 includes performing a publication search based on (i) title, (ii) abstract, (iii) body text, (iv) table captions, (v) table data, (vi) metadata, (vii) figure captions, and/or (iix) figure content. Additionally or alternatively, step 601 may include performing a tabular search based on table attributes. A tabular search may include schema matching (e.g., hierarchical vertical and/or horizontal schema matching, etc.), data transformation and/or unification, processing nested tables inside cells with their own metadata, and/or ranking search-results including such tables by relevance. In various embodiments, the tabular search combines embedding-based schema matching (e.g., tumor size, effect size, size, etc.) and query processing.

At step 612, the system may transmit information associated with the identified node. For example, a user may browse the graph data structure to explore the table clusters attached to the nodes, may select a cluster to query the tables in the cluster, and/or may generate a meta-profile corresponding to the cluster.

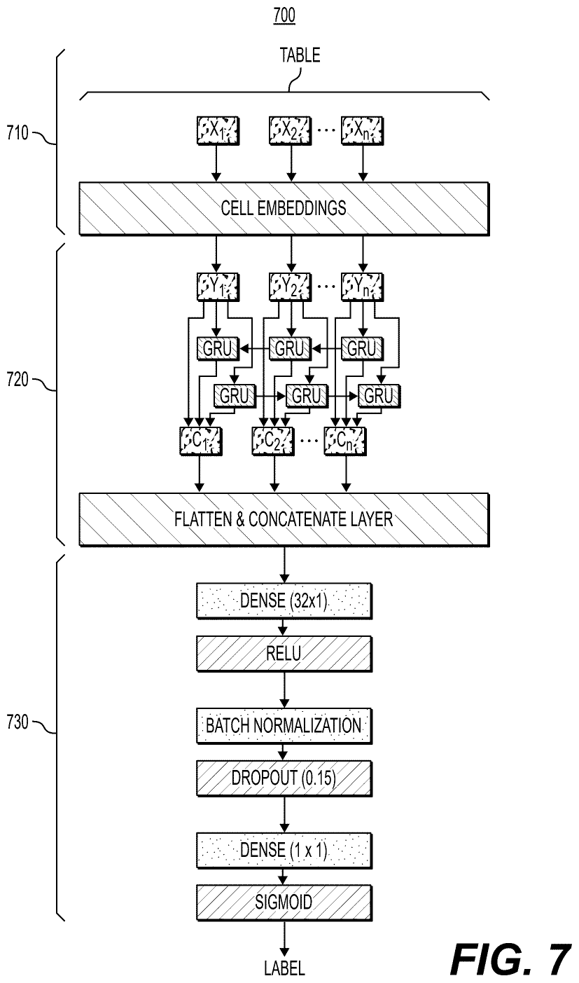

Referring now to FIG. 7, neural network 700 is shown, according to an exemplary embodiment. In various embodiments, processing system 160 implements neural network 700. In various embodiments, neural network 700 ingests data and generates table clusters 180 (e.g., performs topical table classification, etc.). Additionally or alternatively, neural network 700 may generate knowledge graph 120. In various embodiments, neural network 170 is a gated recurrent unit (GRU) network. It should be understood that neural network 700 is described for example purposes only and is not meant to be limiting.

Neural network 700 includes first stage 710, second stage 720, and third stage 730. In first stage 710, a table {x₁, x₂, . . . , xₙ} is pre-processed to create cell-wise representations. First stage 710 may include data cleaning, replacement of numbers and/or ranges (e.g., with placeholders such as NUM, RANGE, etc.). In various embodiments, first stage 710 uses a feature space. For example, first stage may use a 100,000 dimensional feature space. In some embodiments, terms in the feature space are sorted by frequency. In some embodiments, noise words and/or spam words are removed from the feature space. In various embodiments, first stage 710 includes executing one or more regular expressions that encode numerical in categories.

Second stage 720 may include fine-tuning embeddings (e.g., BioBERT embeddings) on the whole corpus. In various embodiments, second stage 720 includes passing the embeddings through a GRU layer and concatenating the result with the original embeddings to create enriched contextualized vectors {c₁, c₂, . . . , cₙ}.

Third stage 730 may include passing the enriched contextualized vectors through a dense layer of units (e.g., 16 units, 32 units, 64 units, etc.), a batch normalization layer, a dropout layer, and/or a dense binary classifier. It should be understood that while neural network 700 has been described with respect to specific architectures, other architectures may be used. For example, LSTM layers may be used instead of GRU layers.

Referring to FIG. 8, an example computing device 800 upon which the methods described herein may be implemented is illustrated. It should be understood that the example computing device 800 is only one example of a suitable computing environment upon which the methods described herein may be implemented. Optionally, computing device 800 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 800 typically includes at least one processing unit 806 and system memory 804. Depending on the exact configuration and type of computing device, system memory 804 may be volatile (such as random access memory (RAM)), non-volatile (such as read-only memory (ROM), flash memory, etc.), or some combination of the two. This most basic configuration is illustrated in FIG. 8 by line 802. Processing unit 806 may be a standard programmable processor that performs arithmetic and logic operations necessary for operation of computing device 800. Computing device 800 may also include a bus or other communication mechanism for communicating information among various components of computing device 800.

Computing device 800 may have additional features/functionality. For example, computing device 800 may include additional storage such as removable storage 808 and non-removable storage 810 including, but not limited to, magnetic or optical disks or tapes. Computing device 800 may also contain network connection(s) 816 that allow the device to communicate with other devices. Computing device 800 may also have input device(s) 814 such as a keyboard, mouse, touch screen, etc. Output device(s) 812 such as a display, speakers, printer, etc. may also be included. The additional devices may be connected to the bus in order to facilitate communication of data among the components of computing device 800. All these devices are well known in the art and need not be discussed at length here.

Processing unit 806 may be configured to execute program code encoded in tangible, computer-readable media. Tangible, computer-readable media refers to any media that is capable of providing data that causes computing device 800 (i.e., a machine) to operate in a particular fashion. Various computer-readable media may be utilized to provide instructions to processing unit 806 for execution. Example tangible, computer-readable media may include, but is not limited to, volatile media, non-volatile media, removable media and non-removable media implemented in any method or technology for storage of information such as computer readable instructions, data structures, program modules or other data. System memory 804, removable storage 808, and non-removable storage 810 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, processing unit 806 may execute program code stored in system memory 804. For example, the bus may carry data to system memory 804, from which processing unit 806 receives and executes instructions. The data received by system memory 804 may optionally be stored on removable storage 808 or non-removable storage 810 before or after execution by processing unit 806.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The term “client or “server” include all kinds of apparatus, devices, and machines for processing data, including by way of example a programmable processor, a computer, a system on a chip, or multiple ones, or combinations, of the foregoing. The apparatus may include special purpose logic circuitry, e.g., a field programmable gate array (FPGA) or an application specific integrated circuit (ASIC). The apparatus may also include, in addition to hardware, code that creates an execution environment for the computer program in question (e.g., code that constitutes processor firmware, a protocol stack, a database management system, an operating system, a cross-platform runtime environment, a virtual machine, or a combination of one or more of them). The apparatus and execution environment may realize various different computing model infrastructures, such as web services, distributed computing and grid computing infrastructures.

The systems and methods of the present disclosure may be completed by any computer program. A computer program (also known as a program, software, software application, script, or code) may be written in any form of programming language, including compiled or interpreted languages, declarative or procedural languages, and it may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry (e.g., an FPGA or an ASIC).

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data (e.g., magnetic, magneto-optical disks, or optical disks). However, a computer need not have such devices. Moreover, a computer may be embedded in another device (e.g., a vehicle, a Global Positioning System (GPS) receiver, etc.). Devices suitable for storing computer program instructions and data include all forms of non-volatile memory, media and memory devices, including by way of example semiconductor memory devices (e.g., EPROM, EEPROM, and flash memory devices; magnetic disks, e.g., internal hard disks or removable disks; magneto-optical disks; and CD ROM and DVD-ROM disks). The processor and the memory may be supplemented by, or incorporated in, special purpose logic circuitry.

To provide for interaction with a user, implementations of the subject matter described in this specification may be implemented on a computer having a display device (e.g., a CRT (cathode ray tube), LCD (liquid crystal display), OLED (organic light emitting diode), TFT (thin-film transistor), or other flexible configuration, or any other monitor for displaying information to the user. Other kinds of devices may be used to provide for interaction with a user as well; for example, feedback provided to the user may be any form of sensory feedback (e.g., visual feedback, auditory feedback, or tactile feedback).

Implementations of the subject matter described in this disclosure may be implemented in a computing system that includes a back-end component (e.g., as a data server), or that includes a middleware component (e.g., an application server), or that includes a front end component (e.g., a client computer) having a graphical user interface or a web browser through which a user may interact with an implementation of the subject matter described in this disclosure, or any combination of one or more such back end, middleware, or front end components. The components of the system may be interconnected by any form or medium of digital data communication (e.g., a communication network). Examples of communication networks include a LAN and a WAN, an inter-network (e.g., the Internet), and peer-to-peer networks (e.g., ad hoc peer-to-peer networks).

What is claimed is:

1. A method comprising:
displaying, on a graphical user interface, a knowledge graph associated with a domain, wherein the knowledge graph comprises a plurality of nodes and a plurality of edges representing relationships between the plurality of nodes, wherein the plurality of nodes comprise a plurality of leaf nodes, each of the plurality of leaf nodes being associated with respective metadata related to the domain;
receiving, at the graphical user interface, one or more user inputs, wherein the one or more user inputs comprise a selection of a specific leaf node of the plurality of leaf nodes;
displaying, on the graphical user interface, the respective metadata related to the domain that is associated with the specific leaf node; and
providing, on the graphical user interface, a search window configured to receive a search query related to the domain.

2. The method of claim 1, further comprising:
receiving, at the search window, the search query;
executing, using a search engine, the search query against the respective metadata related to the domain that is associated with the specific leaf node; and
returning, in response to the search query, a subset of the respective metadata related to the domain that is associated with the specific leaf node.

3. The method of claim 2, wherein the search engine is a keyword search engine.

4. The method of claim 2, wherein the search engine is a structural search engine.

5. The method of claim 2, wherein the search engine is a large language model (LLM) search engine.

6. The method of claim 1, further comprising generating a three-dimensional (3D) meta-profile for the respective metadata related to the domain that is associated with the specific leaf node.

7. The method of claim 6, further comprising displaying, on the graphical user interface, the 3D meta-profile.

8. The method of claim 1, further comprising constructing the knowledge graph.

9. The method of claim 8, wherein constructing the knowledge graph comprises:
initializing a structural hierarchy of the knowledge graph based, at least in part, on a user specification; and
automatically fusing the respective metadata related to the domain to each of the plurality of leaf nodes.

10. The method of claim 1, wherein the domain is cancer.

11. A system comprising:
a computing cluster comprising a plurality of computing devices, each computing device comprising at least one processor and a memory operably coupled to the at least one processor; and
a database operably coupled to the computing cluster, wherein the computing cluster is configured to:
display, on a graphical user interface, a knowledge graph associated with a domain, wherein the knowledge graph comprises a plurality of nodes and a plurality of edges representing relationships between the plurality of nodes, wherein the plurality of nodes comprise a plurality of leaf nodes, each of the plurality of leaf nodes being associated with respective metadata related to the domain;
receive, at the graphical user interface, one or more user inputs, wherein the one or more user inputs comprise a selection of a specific leaf node of the plurality of leaf nodes;
display, on the graphical user interface, the respective metadata related to the domain that is associated with the specific leaf node; and
provide, on the graphical user interface, a search window configured to receive a search query related to the domain.

12. A non-transitory computer-readable storage medium, having instruction stored thereon that, when executed by a processor, cause the processor to:
initialize a graph data structure using a seed to generate an initialized graph data structure;
train a machine learning (ML) model using a corpus to generate a hierarchical data structure comprising a subtree extracted from the corpus; and
update the initialized graph data structure using the hierarchical data structure by adding at least one of (i) a node or (ii) an edge to the initialized graph data structure to generate an updated graph data structure, wherein the node or the edge is a representation of at least a portion of the subtree.

13. The non-transitory computer-readable storage medium of claim 12, wherein generating the hierarchical data structure comprises analyzing the corpus using a large language model (LLM) to produce a topical table cluster, wherein the subtree is associated with a cluster of the topical table cluster.

14. The non-transitory computer-readable storage medium of claim 13, wherein analyzing the corpus comprises:
generating at least two embedding vectors based on the corpus;
generating a centroid vector based on the initialized graph data structure; and
comparing the at least two embedding vectors to the centroid vector to identify an embedding vector of the at least two embedding vectors that is within a threshold degree from the centroid vector.

15. The non-transitory computer-readable storage medium of claim 14, wherein the threshold degree is 18 degrees.

16. The non-transitory computer-readable storage medium of claim 12, wherein the corpus is represented in a JavaScript Object Notation (JSON) format.

17. The non-transitory computer-readable storage medium of claim 12, wherein updating the initialized graph data structure using the hierarchical data structure comprises identifying a node of the initialized graph data structure that corresponds to a node of the subtree.

18. The non-transitory computer-readable storage medium of claim 12, wherein the instructions further cause the processor to:
receive a query for information;
traverse the updated graph data structure to identify a node associated with the query; and
transmit information associated with the identified node.

19. The non-transitory computer-readable storage medium of claim 18, wherein transmitting the information associated with the identified node comprises displaying a graphical user interface (GUI) that represents the identified node.

20. The non-transitory computer-readable storage medium of claim 18, wherein receiving the query comprises performing natural language processing (NLP) on a string.
